(19) United States
(12) Patent Application Publication
(10) Pub. No.: US 2012/0296408 A1
(43) Pub. Date: Nov. 22, 2012

(54) METHODS AND SYSTEMS FOR PERFORMING VASCULAR RECONSTRUCTION

(76) Inventors: Donald K. Jones, Dripping Springs, TX (US); Vladimir Mitelberg, Austin, TX (US)

(21) Appl. No.: 13/574,720

(22) PCT Filed: Jan. 24, 2011
(86) PCT No.: PCT/US11/22255

§ 371 Date: Jul. 23, 2012

Related U.S. Application Data
(60) Provisional application No. 61/298,060, filed on Jan. 25, 2010.
(60) Provisional application No. 61/298,046, filed on Jan. 25, 2010.

(51) Int. Cl. A61F 2/84; A61F 2/82

(52) U.S. Cl. 623/1.11; 623/1.15

(57) ABSTRACT

Devices, systems and methods are provided for performing intra-lumenal medical procedures in a desired area of the body. Stents, stent delivery devices and methods of performing medical procedures to redirect and or re-establish the intravascular flow of blood are provided for the treatment of hemorrhagic and ischemic disease states.

### FIELD OF THE INVENTION

The present invention relates to methods and systems for performing intralumenal procedures including vascular reconstruction. More particularly the present invention relates to systems utilizing stents, stent delivery devices and methods of performing medical procedures to redirect and or re-establish the intravascular flow of blood.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

For instance, one aspect of cerebrovascular disease in which the wall of a blood vessel becomes weakened. Under cerebral flow conditions the weakened vessel wall forms a bulge or aneurysm which can lead to symptomatic neurological deficits or ultimately a hemorrhagic stroke when ruptured. Once diagnosed a small number of these aneurysms are treatable from an endovascular approach using various embolization devices. These embolization devices include detachable balloons, coils, polymerizing liquids, gels, foams, stents and combinations thereof.

The most widely used embolization devices are detachable embolization coils. These coils are generally made from biologically inert platinum alloys. To treat an aneurysm, the coils are navigated to the treatment site under fluoroscopic visualization and carefully positioned within the dome of an aneurysm using sophisticated, expensive delivery systems. Typical procedures require the positioning and deployment of multiple embolization coils which are then packed to a sufficient density as to provide a mechanical impediment to flow impingement on the fragile diseased vessel wall. Some of these bare embolization coil systems have been describe in U.S. Pat. No. 5,108,407 to Geremia, et al., entitled, “Method And Apparatus For Placement Of An Embolic Coil” and U.S. Pat. No. 5,122,136 to Guglielmi, et al., entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils at predetermined positions within vessels of the human body in order to treat aneurysms, or alternatively, to occlude the blood vessel at a particular location. Many of these systems, depending on the particular location and geometry of the aneurysm, have been used to treat aneurysms with various levels of success. One drawback associated with the use of bare embolization coils relates to the inability to adequately pack or fill the aneurysm due to the geometry of the coils which can lead to long term recanalization of the aneurysm with increased risk of rupture.

Some improvements to bare embolization coils have included the incorporation of expandable foams, bioactive materials and hydrogel technology as described in the following U.S. Pat. No. 6,723,108 to Jones, et al., entitled, “Foam Matrix Embolization Device”, U.S. Pat. No. 6,423,085 to Murayama, et al., entitled, “Biodegradable Polymer Coils for Intraluminal Implants” and U.S. Pat. No. 6,238,403 to Greene, et al., entitled, “Filamentous Embolic Device with Expansible Elements.” While some of these improved embolization coils have been moderately successful in preventing or reducing the rupture and re-rupture rate of some aneurysms, the devices have their own drawbacks. For instance, in the case of bioactive coils, the materials eliciting the biological healing response are somewhat difficult to integrate with the coil structure or have mechanical properties incompatible with those of the coil making the devices difficult to accurately position within the aneurysm. In the case of some expandable foam and hydrogel technology, the expansion of the foam or hydrogel is accomplished due to an interaction of the foam or hydrogel with the surrounding blood environment. This expansion may be immediate or time delayed but is generally, at some point, out of the control of the physician. With a time delayed response the physician may find that coils which were initially placed accurately and detached become dislodged during the expansion process leading to subsequent complications.

For many aneurysms, such as wide necked or fusiform aneurysms the geometry is not suitable for coiling alone. To somewhat expand the use of embolization coils in treating some wide necked aneurysms, stent like scaffolds have been developed to provide support for coils. These types of stent like scaffolds for use in the treatment of aneurysms have been described in U.S. Pat. No. 6,605,111 to Bose et al., entitled, “Endovascular Thin Film Devices and Methods for Treating Strokes” and U.S. Pat. No. 6,673,106 to Mitelberg, et al., entitled, “Intravascular Stent Device”. While these stent like devices have broadened the types of aneurysms amenable to embolization therapy, utilization of these devices in conjunction with embolization devices is technically more complex for the physician, may involve more risk to the patient and have a substantial cost increase for the healthcare system.

To further expand the types of aneurysm suitable for interventional radiological treatment, improved stent like devices have been disclosed in U.S. Pat. No. 5,824,053 to Khosravi et al., entitled, “Helical Mesh Endoprosthesis and Method”, U.S. Pat. No. 5,951,599 to McCrory, entitled, “Occlusion System for the Endovascular Treatment of and Aneurysm” and U.S. Pat. No. 6,063,111 to Hieshima et al., entitled, “Stent Aneurysm Treatment System and Method.” When placed across the neck of an aneurysm the proposed stent like devices purport to have a sufficient density through the wall of the device to reduce flow in the aneurysm allowing the aneurysm to clot, while at the same time having a low enough density through the wall to allow small perforator vessels adjacent to the aneurysm to remain patent. Stent devices of this nature while having the potential to reduce treatment costs have not been realized commercially due to the difficulty in manufacturing, reliability in delivering the devices to the treatment site and an inability to properly position the denser portion of the stent device accurately over the neck of the aneurysm.

Another cerebrovascular disease state is ischemia resulting from reduced or blocked arterial blood flow. The arterial blockage may be due to thrombus, plaque, foreign objects or a combination thereof. Generally, soft thrombus created elsewhere in the body (for example due to atrial fibrillation) that lodges in the distal cerebrovasculature may be disrupted or dissolved using mechanical devices and or thrombolytic drugs. While guidewires are typically used to disrupt the thrombus, some sophisticated thrombectomy devices have been proposed. For instance U.S. Pat. No. 4,762,130 to Fogarty et al., entitled, “Catheter with Corkscrew-Like Balloon”, U.S. Pat. No. 4,998,919 of Schepp-Pesh et al., entitled, “Thrombectomy Apparatus”, U.S. Pat. No. 5,417,703 to Brown et al., entitled “Thrombectomy Devices and Methods of Using Same”, and U.S. Pat. No. 6,663,650 to Sepetka et al., entitiled, “Systems, Methods and Devices for Removing Obstructions from a Blood Vessel” discloses devices such as catheter based corkscrew balloons, baskets or filter wires and helical coiled retrievers. Commercial and prototype versions of these devices have shown only marginal improvements over guidewires due to an inability to adequately grasp the thrombus or to gain vascular access distal to the thrombus(i.e. distal advancement of the device pushes the thrombus distally).

Plaque buildup within the lumen of the vessel, known as atherosclerotic disease, is not generally responsive to thrombolytics or mechanical disruption using guidewires. The approach to the treatment of neurovascular atherosclerotic disease has been to use modified technology developed for the treatment of cardiovascular atherosclerotic disease, such as balloons and stents, to expand the vessel at the site of the lesion to re-establish blood flow. For instance, U.S. Pat. No. 4,768,507 to Fischell et al., entitled, “Intravascular Stent and Percutaneous Insertion Catheter System for the Dilation of an Arterial Stenosis and the Prevention of Arterial Restenosis” discloses a system used for placing a coil spring stent into a vessel for the purposes of enhancing luminal dilation, preventing arterial restenosis and preventing vessel blockage resulting from intimal dissection following balloon and other methods of angioplasty. The coil spring stent is placed into spiral grooves on an insertion catheter. A back groove of the insertion catheter contains the most proximal coil of the coil spring stent which is prevented from springing radially outward by a flange. The coil spring stent is deployed when an outer cylinder is moved proximally allowing the stent to expand. Other stent systems include those disclosed in U.S. Pat. No. 4,512,338 to Balko, et al., entitled, “Process for Restoring Patency to Body Vessels”, U.S. Pat. No. 5,354,309 to Schnepp Pesch et al., entitled, “Apparatus for Widening a Body Cavity” and U.S. Pat. No. 6,833,003 to Jones et al., entitled, “Expandable Stent and Delivery System”. While the aforementioned devices may have the ability to access the cerebrovasculature, they lack sufficient structural coverage of the lesion to achieve the desired patency of the vessel without the use of a balloon device.

### SUMMARY OF THE INVENTION

In accordance with one aspect of the present invention there is provided a medical device deployment system for repairing a body lumen in a mammal. The medical device deployment system includes a stent device, a delivery member and a catheter. The stent device is positioned at the distal end of the delivery member and disposed within the lumen of the catheter. The stent device takes the form of a helically wound wire backbone or primary member having side extension members spaced apart along the length and extending outwardly from the backbone. The side extension members generally have two ends where one end is fixedly coupled to the backbone and the other end extending from the backbone is free, meaning it is typically uncoupled to any other structural member. As the backbone takes successive helical turns, the side extension members may be positioned adjacent to or overlap the side extension members or backbone of subsequent or previous helical turns, generally forming a tubular structure. The overlapping side extension members create a lattice work of apertures between turns of the backbone. The size and distribution of the apertures is a function of the diameter, length and shape of the side extension members and the distance between turns of the backbone. The stent device is formed of a resilient material and has a first constrained elongate tubular configuration for delivery to a target site within a body lumen and a second unconstrained expanded tubular configuration for deployment at the target site. The delivery member distal end includes a helical retainer wall secured to the delivery member which defines a helical gap between successive turns of the retainer wall. The stent device is mounted on the distal end of the delivery member where turns of the stent device are positioned within the turns of the helical gap. The mounted stent device is positioned within the catheter lumen where the catheter wall constrains the stent device and maintains the stent device within the helical gap.

In accordance with another aspect of the present invention there is provided a stent device having a backbone and side extension members which may take various configurations comprising any of the following: side extension members on each side of the backbone which are uniformly spaced along the length of the backbone; side extension members on each side of the backbone which are not uniformly spaced along the length of the backbone; side extension members having a curved shape; side extension members having a straight shape; side extension members extending from the backbone in an angled direction; side extension members having different lengths; side extension members having apertures; side extension members having radio-opaque markers; side extensions having an enlarged tabular end; backbones having apertures; backbones having radio-opaque marker(s); backbones having a curvilinear shape.

In accordance with still another aspect of the present invention there is provided a method of reconstructing a body lumen having a defect using a stent device according to an embodiment of the present invention. The method comprises the steps of: positioning a stent device deployment system within a vessel adjacent a target site; retracting the catheter relative to the delivery member, thereby allowing a portion of the stent to be deployed adjacent the target site; controlling the amount of overlap of the side extension members of the stent device during deployment of the stent adjacent the target site; releasing the stent device from the delivery member distal end and catheter lumen.

In accordance with still another aspect of the present invention there is provided a method of reconstructing a body lumen having a defect, such as an aneurysm, using a stent device according to an embodiment of the present invention in conjunction with embolization devices, such as embolic coils. The method comprises the steps of: providing a stent device having a configuration adapted to receive an embolization catheter through the side wall of the stent when said stent is in a deployed configuration; positioning a stent device deployment system having a delivery member and a catheter within a vessel adjacent a target site; retracting the catheter relative to the delivery member, thereby allowing a portion of the stent to be deployed adjacent the target site; controlling the amount of overlap of the side extension members of the stent device during deployment of the stent adjacent the target site; releasing the stent device from the delivery member distal end and catheter lumen; positioning an embolization catheter through the wall of the deployed stent; delivering embolization devices to the aneurysm wherein said embolization devices are supported by the stent device; releasing said embolization devices.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second ends where one of the first and second ends is fixedly coupled to the primary member and the other end is uncoupled to any other member of said reconstruction device.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second ends and a body portion between said ends, where one of the first and second ends is fixedly coupled to the primary member and the body portion or other end is uncoupled to any other member of said reconstruction device that interconnects with said backbone.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second end portions and a body portion between said end portions, where one of the first and second end portions is fixedly coupled to the primary member and the body portion or other end is uncoupled to any other member of said reconstruction device that interconnects with said backbone.

In accordance with still yet another aspect of the present invention there is provided a reconstruction device wherein the primary helical member is formed of a resilient non-absorbable non-erodible material and a plurality of the extension members are formed of an absorbable or bio-erodible material.

In accordance with still yet another aspect of the present invention there is provided a reconstruction device wherein the primary helical member is formed of a resilient material and includes an absorbable and or erodible material and a plurality of the extension members are formed of a resilient material and includes an absorbable and or erodible material.

In accordance with yet still another aspect of the present invention there is provided a reconstruction device comprising a biocompatible material. Suitable resilient materials include metal alloys such as Nitinol(NiTi), titanium, chromium alloy, stainless steel. Additional materials include polymers such as polyolefins, polyimides, polyamides, fluoropolymers, polyetheretherketone(PEEK), cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials such as polylactide polymers and polyglycolide polymers or copolymers thereof and shape memory polymers. The medical device may comprise numerous materials depending on the intended function of the device. These materials may be formed into desired shapes or attached to the device by a variety of methods which are appropriate to the materials being utilized such as laser cutting, injection molding, spray coating and casting.

In accordance with another aspect of the present invention there is provided a reconstruction device having a coating formed of a biocompatible, bioerodible and biodegradable synthetic material. The coating may further comprise one or more pharmaceutical substances or drug compositions for delivering to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the medical device.

In accordance with another aspect of the present invention there is provided a delivery member where the delivery member distal end includes a helical retainer wall secured to the delivery member which defines a helical gap between successive turns of the retainer wall. The distal end of the delivery member takes the form of a flexible, torque-able construction such that rotation of the proximal end of the delivery member causes the distal end of the delivery member and the retainer wall to rotate.

In accordance with yet another aspect of the present invention there is provided a delivery member which includes a tip marker at its distal end and a stent positioning marker proximal to the tip marker.

In accordance with still yet another aspect of the present invention there is provided an attachable handle assembly having a housing member, a catheter coupler and a rotatable delivery member coupler which couples to both the proximal end of the delivery catheter and the proximal end of the delivery member. The handle assembly further includes an actuator member that takes the form of a rotatable deployment knob connected to a gear assembly which is also coupled to the rotatable delivery member coupler such that rotation of the deployment knob causes the rotation of the rotatable delivery member coupler. The gear assembly may be configured such that one rotation of the deployment knob corresponds to multiple rotations of the rotatable delivery member coupler. The rotatable delivery member coupler may be selectively secured to the proximal end of the delivery member such that rotation of the deployment knob causes rotation of the delivery member.

In accordance with still another aspect of the present invention there is provided a method of reconstructing a body lumen having a defect using a stent device according to an embodiment of the present invention. The method comprises the steps of: positioning a stent device deployment system within a vessel wherein the positioning marker of the delivery member is adjacent a target site; retracting the catheter relative to the delivery member, such that the tip marker of the delivery member extends distal to a catheter tip marker, thereby allowing a portion of the stent to be deployed adjacent the target site; rotating the distal end of the delivery member and retainer wall such that the stent device further deploys from the catheter lumen; controlling the amount of overlap of the side extension members of the stent device during deployment of the stent adjacent the target site; disengaging the stent device from the delivery member distal end and catheter lumen.

In accordance with yet still another aspect of the present invention there is provided a method of reconstructing a body lumen having a defect using a stent device further including the steps of: connecting a handle assembly to the proximal end of the catheter and the delivery member; manipulating a deployment knob on the handle assembly to cause the rotation of the distal end the delivery member.

### BRIEF DESCRIPTION OF THE DRAWINGS

FIG. 1 is a partial cross-sectional of a stent deployment system according to an embodiment of the present invention.

FIG. 2 is an enlarged partial cross-sectional view of the distal end of the stent deployment system according to an embodiment of the present invention.

FIG. 3A is a side view of a deployed stent device according to an embodiment of the present invention.

FIG. 3B is a side view of a deployed stent device according to another embodiment of the present invention.

FIGS. 4A through 4L are partial flat pattern views of stent devices according to embodiments of the present invention.

FIGS. 5A through 5C are partial cross-sectional views illustrating a method of delivering and deploying a stent device within a vessel at a target site adjacent an aneurysm according to an embodiment of the present invention.

FIGS. 6A and 6B are partial cross-sectional views illustrating a method of delivering and deploying a stent device within a vessel at a target site adjacent an aneurysm according to another embodiment of the present invention.

FIGS. 7A through 7E are partial cross-sectional views illustrating a method of delivering and deploying a stent device within a vessel at a target site adjacent an atherosclerotic lesion according to an embodiment of the present invention.

FIG. 8 is a partial cross-sectional of a stent deployment system having an improved delivery system according to an embodiment of the present invention.

FIGS. 9A and 9B are enlarged partial cross-sectional view of the distal end of the stent deployment system having an improved delivery system according to an embodiment of the present invention.

FIG. 10 is an enlarged partial cross-sectional top view of the proximal end of the stent deployment system including a handle assembly according to an embodiment of the present invention.

FIGS. 11A through 11D are partial cross-sectional views illustrating a method of delivering and deploying a stent device within a vessel at a target site adjacent an aneurysm according to an embodiment of the present invention.

FIG. 12A is a side view of a deployed stent device where the side extension members of subsequent helical turns do not overlap according to an embodiment of the present invention.

FIG. 12B is a side view of a deployed stent device where the side extension members of subsequent helical turns overlap the adjacent backbone according to another embodiment of the present invention.

FIGS. 13A through 13D are partial cross-sectional views illustrating a method of delivering and deploying a stent device and embolization devices within a vessel at a target site according to an embodiment of the present invention.

### DETAILED DESCRIPTION OF THE INVENTION

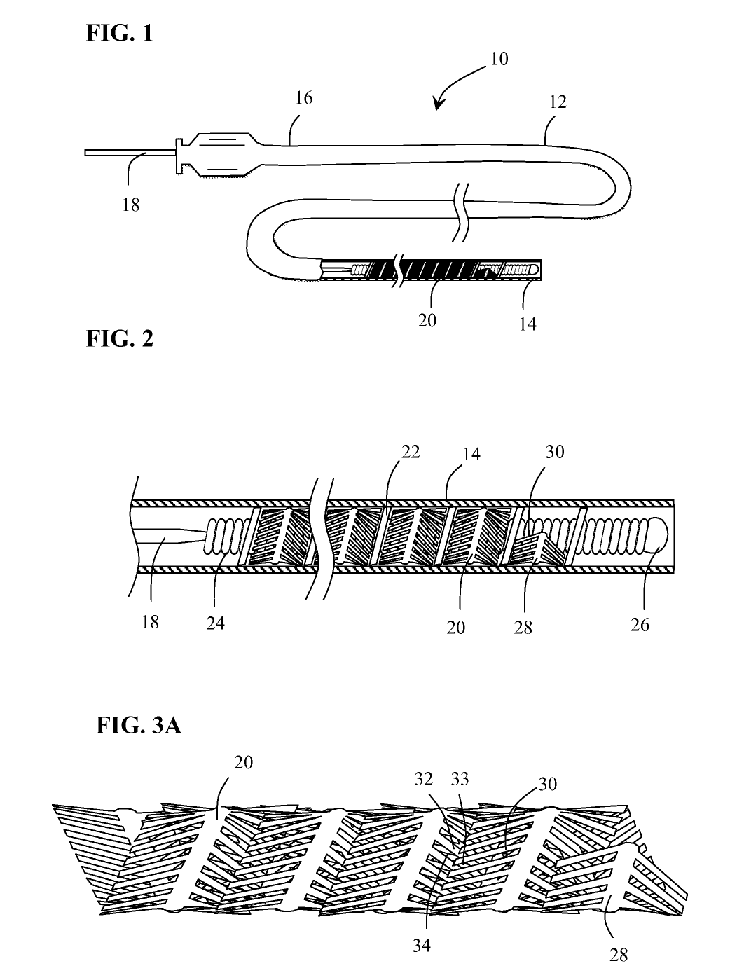

Methods and systems for performing vascular reconstruction and revascularization in a desired area of the body are herein described. FIG. 1 illustrates a medical device deployment system 10 suitable for use with embodiments of the present invention. System 10 includes a catheter 12 having distal and proximal ends 14 and 16 respectively, an elongate delivery member 18, and a stent device 20 for use in vascular reconstruction or revascularization procedures.

FIG. 2 depicts a magnified partial cross-sectional view of catheter distal end 14. Slidably positioned within the lumen of catheter 12 is delivery member 18. Delivery member 18 is preferably formed as an elongate wire assembly and includes at its distal end a raised spiral retainer member 22 secured to a flexible coil 24. Located at the distal end of coil 24 is an atraumatic tip 26. As shown, atraumatic tip 26 may be formed as a rounded bead using beading processes known for guide wire manufacturing such as soldering, plasma or arc welding. Alternatively atraumatic tip 26 may be formed of a soft polymer extension to reduce the likelihood of vessel perforation. Spiral retainer member 22 preferably takes to form of a flexible wire which is fixedly secured to the outer surface coil 24 using known processes such as soldering welding or gluing. The pitch of spiral retainer member 22 creates a spiral gap relative to the outer surface of coil 24 and the wall of catheter distal end 14. Also located at the distal end of delivery member 18 is stent device 20 which is generally formed as a helix includes a primary backbone 28 having a plurality of side extension members 30. Stent device 20 has a first constrained configuration for delivery to a target location and a second expanded configuration for deployment at a target site. In the first constrained configuration of stent device 20, primary backbone 28 and side extension members 30 are positioned within the spiral gap formed by spiral retainer member 22 and are constrained by the inner wall of catheter distal end 14.

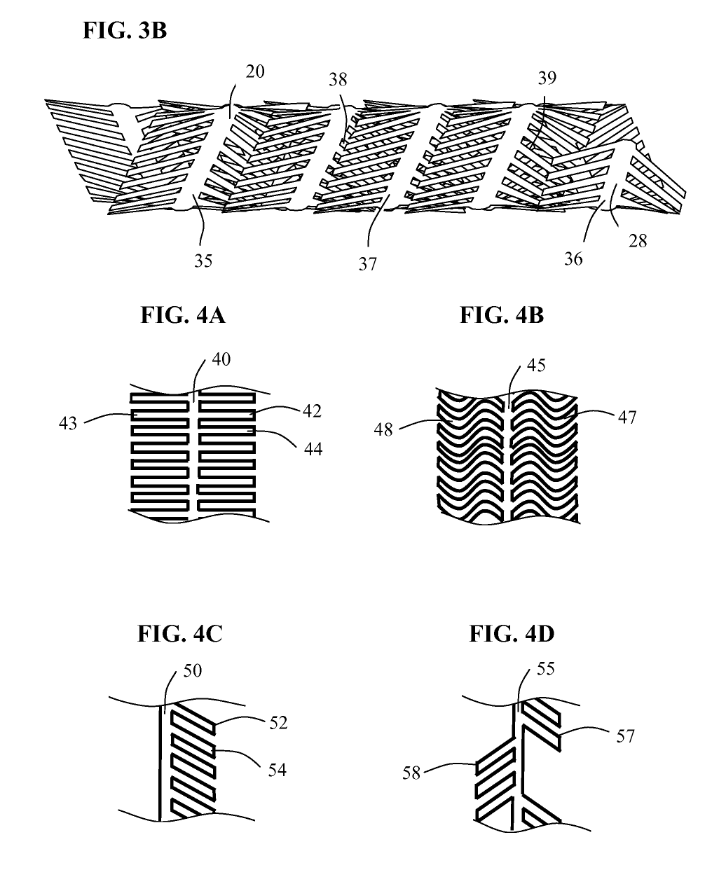

FIG. 3A illustrates detail of stent device 20 in an expanded configuration. The primary backbone 28 is shown in a uniform helical shape along with side extension members 30 and adjacent turn side extension members 32. As depicted, the side extension members of the stent device generally have one end secured to the backbone and the other end uncoupled which is unlike previous stents described in the art. This configuration allows the side extension members of the present invention to act as individual cantilevers providing an improved ability to conform to discrete contours within the vasculature. Alternatively, both ends of the side extension members may be coupled to the primary backbone, forming a looped structure for example, as long as the side extension member is discrete and not fixedly coupled to any other structural member. Prior art helical stents formed of a ladder or mesh structure in which side extension members do not have a free end or are not discrete, such as those described in U.S. Pat. No. 6,660,032 to Klumb et al, entitled, “Expandable Coil Endoluminal Prosthesis” or U.S. Pat. No. 5,824,053 to Koshravi et al, entitled “Helical Mesh Endoprosthesis and Method of Use”, do not have the same ability to conform to discrete contours of a lesion within the vasculature and instead form a wide area “tented” surface. Stent device 20 is shown with side extension members 30 partially overlapping adjacent turn side extension members 32. The overlap of these side extension members creates overlap apertures 33 and residual apertures 34. The size, shape and distribution of the overlap apertures 33 and the residual apertures 34 is dependant upon the size shape and distribution of the side extension members 30 and adjacent turn side extension members 32 and the degree of overlap of the adjacent turns defined in part by the pitch of the primary backbone 28. The stent may have a diameter in the range of 1 to 50 mm, and preferably between 2 and 15 mm. The diameters of the side extension members have a range of between 0.0001 and 0.025 inches with a preferred range of between 0.002 to 0.010 inches. The spacing between side extension members range between 0.001 and 0.250 inches with a preferred range between 0.002 and 0.060 inches. FIG. 3B illustrates detail of stent device 20 according to another embodiment of the present invention wherein backbone 28 has a non-uniform helical shape in which the pitch at stent ends 35 and 36 is wider than the pitch towards the stent middle 37. This pitch variation causes the overlap of side extension members near the ends of the stent to be less than the overlap of side extension members towards the middle of the stent. As an outcome of the variable overlap there is a larger number of smaller overlap apertures in the middle of the stent than at the stent ends and the residual aperture 38 adjacent the stent middle 37 is substantially smaller than the residual aperture 39 adjacent the stent ends 35 and 36. The ensuing result is that the stent device 20 has a lower porosity in the stent middle 37 and a higher porosity at the stent ends 35 and 36.

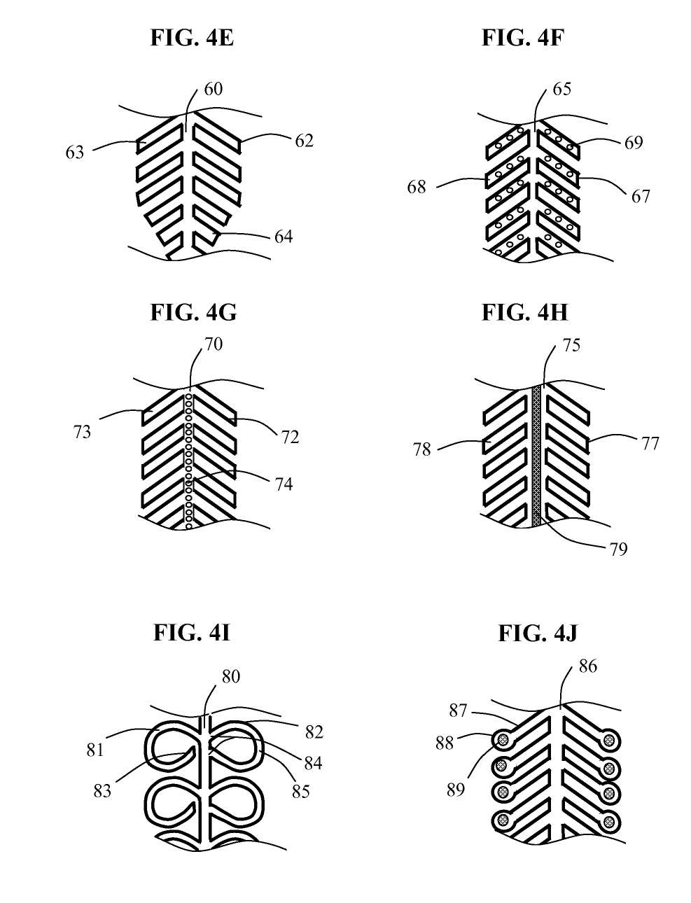

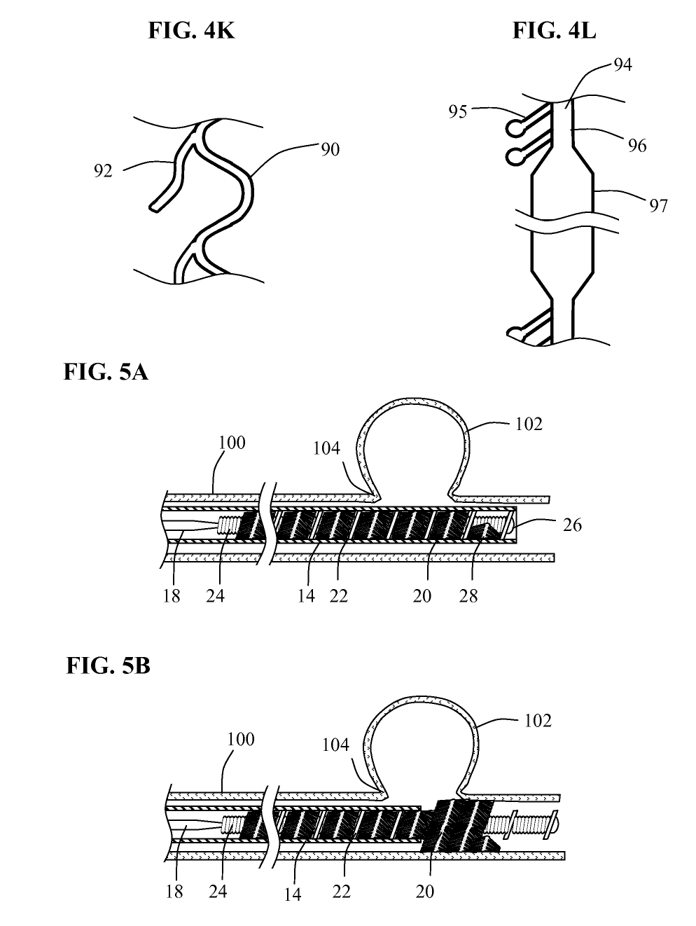

In addition to the pitch of the stent backbone having an influence on the overall porosity and porosity distribution of the stent device there exists numerous variations in the size shape and distribution of side extension members that may also influence porosity. FIGS. 4A through 4E illustrate partial flat patterns of some variations of side extension members relative to a backbone that may affect different aspects of stent performance including porosity and porosity distribution when formed in a helical shape. In one pattern variation shown in FIG. 4A, a stent device has a primary backbone 40 with side extension members 42 and 43, having generally similar diameters and lengths, extending from opposite sides of the backbone 40. Side extension member 44, positioned adjacent extension member 42 has a similar length to extension 42 however may have a smaller diameter. The alternating pattern of side extension members having different diameters may be extended along backbone 40. FIG. 4B depicts another pattern variation in which a stent device has a primary backbone 45 and side extension members 47 and 48, with generally similar diameters and lengths extending from opposite sides of backbone 45 in a curvilinear shape. FIG. 4C illustrates another pattern variation in which a stent device has a primary backbone 50 and side extension members 52 and 54 which are positioned on only one side of the backbone 50. FIG. 4D shows still another pattern variation in which a stent device has a primary backbone 55 and groups of side extension members 57 and 58 are positioned in an alternating configuration on opposite sides of the backbone. FIG. 4E depicts still another pattern variation in which a stent device has a primary backbone 60 and side extension members 62 and 63 with generally similar diameters and lengths extending from opposite sides of backbone 60. Additionally, the side extension members may progressively have shorter lengths, such as side extension member 64, to provide a tapered configuration. FIG. 4F illustrates yet still another pattern in which a stent device has a primary backbone 65 and side extension members, 67 and 68 with generally similar diameters and lengths extending from opposite sides of backbone 65. Additionally the side extension members contain apertures 69. FIGS. 4G and 4H illustrate partial flat patterns of some variations of a backbone relative to side extension members that may affect different aspects of stent performance including porosity and porosity distribution as well as radiographic visibility when formed in a helical shape. FIG. 4G depicts a pattern of a stent device that has a primary backbone 70 and side extension members 72 and 73, with generally similar diameters and lengths extending from opposite sides of backbone 70. Along the length of backbone 70 there is a plurality of apertures 74. FIG. 4H depicts a pattern of a stent device that has a primary backbone 75 and side extension members 77 and 78, with generally similar diameters and lengths extending from opposite sides of backbone 75. Along the length of backbone 75 there is a radio-opaque member 79. The radio-opaque member 79 provides fluoroscopic visualization of the stent during the deployment procedure. For a stent device having an expanded diameter and a pre-set initial overlap of side extension members upon each helical turn of the backbone, the radio-opaque member 79 provides a visual indication of the stent pitch. As the spacing between adjacent turns of radio-opaque member 79 decreases, the amount of side extension member overlap with adjacent turns increases. FIG. 4I depicts yet another pattern of a stent device that has a primary backbone 80 and a plurality of side extension members represented by side extension members 81 and 82. Side extension members 81 and 82 are positioned on opposite sides of backbone 80 in a generally mirrored fashion for this configuration. Side extension members 81 generally take the form of an open ended loop, where a first end of the extension member loop is connected to the backbone and the second end 83 is adjacent the backbone but are not connected. Side extension members 82 generally take the form of a closed loop, where two ends 84 of the extension member loop are connected to backbone 80. As can be appreciated, side extension members 82 form a discrete side unit where it is unconnected to other side extension members except through the backbone 80. From a broader perspective two ends 84 may be considered as a first end region coupled to the backbone and a second end region 85, as shown, is free or uncoupled to any other structural member. While these loops are shown generally “circular”, the size and shape of the loop may take the form of other geometric shapes and patterns to be commensurate with the desired properties of the formed stent. For instance the loops may be rectangular, triangular or form a flattened spiral. FIG. 4J illustrates another pattern of a stent device according to an embodiment of the present invention that has a primary backbone 86 and a representative side extension member 87. While a first end of side extension member 87 is integrally coupled to backbone 86, the second end of the side extension member is uncoupled to the backbone and takes the form of an enlarged tabular end 88. This tabular end 88 is preferably rounded as to be atraumatic to the vessel wall and may include a marker element 89. Preferably marker element 89 is radio-opaque for use in fluoroscopy using known materials such as gold, platinum, tantalum, tungsten, etc., however marker materials suitable for direct visual or magnetic resonance imaging are also contemplated. Marker element 89 may be formed using coining techniques in which a round marker is press fit into a slightly smaller opening positioned on tabular end 88. Alternatively, marker 89 may be printed, coated, electro-deposited, riveted, glued, recessed or raised relative to tabular end 88. More broadly, an entire stent device or portion thereof may be coated with a radio-opaque material to provide visibility under fluoroscopy. While the marker shown in FIG. 4J is positioned at tabular end 88, the marker may be positioned at any location on the side extension member. For instance the side extension member may take the form of a threaded member and a marker take the form of a coil that is wound over the side extension member. FIG. 4K depicts still yet another flat pattern of a stent device in which the backbone 90 takes a curvilinear shape. For representative simplicity, backbone 90 is shown as being somewhat sinusoidal. Side extension member 92, also shown to be curvilinear, extends from a peak on backbone 90. As can be appreciated, side extension members such as side extension member 92 may extend from different locations on backbone 90. FIG. 4L illustrates a stent pattern where backbone 94 has side extension members represented by side extension member 95. Along its length, backbone 94 has a first width 96 and a second width 97. To impart some stretch resistance for the finished stent width 97 is shown to be greater than width 96. The amount of stretch resistance imparted in the finished stent is related to the relative difference between the two widths. The larger width may range from 1.01 to 100 times the width of the smaller width with a preferable range of 1.5 to 20 times. While FIG. 4L shows two such differing widths of the backbone, a stent may have multiple regions of differing width to make the stent suitable for a particular anatomy and clinical application. As with any of the aforementioned stent device pattern variations, these patterns may extend along the entire length of the backbone or only a portion thereof and in some instances features of various patterns may be provided in a combined fashion to form stent devices having unique performance characteristics. Preferably stent devices of the present invention comprise a biocompatible resilient material. Suitable resilient materials include metal alloys such as nitinol, titanium, stainless steel. Additional suitable materials include polymers such as polyimides, polyamides, fluoropolymers, polyetheretherketone(PEEK) and shape memory polymers. As can be appreciated, embodiments of stent devices of the present invention may be formed in part or entirely of bioabsorbable and or bioerodible materials such as polycaprolactone (PCL), polyglycolic acid (PGA), polydioxanone (PDO) and combinations thereof to allow the stent to temporarily serve structural clinical applications, deliver pharmacological compounds and then dissolve over time. These materials may be formed into desired shapes by a variety of methods which are appropriate to the materials being utilized such as laser cutting, thermal heat treating, vacuum deposition, electro-deposition, vapor deposition, chemical etching, photo-chemical etching, electro etching, stamping, injection molding, casting or any combination thereof. Preferably the stent backbone and the side extension members are integrally formed. The distance a side extension member extends from the backbone is dependant upon a specific stent design but a typical range includes between 0.5 to 100 times the width of the backbone and a preferred range being about 0.75 to 25 times the backbone width. The backbone widths have a general range of about 0.0005 in to 0.250 in with a preferred range of about 0.001 in to 0.100 in. While various configurations of side extension members, backbones and a discussion of pitch have been provided, the features of a particular stent design features are heavily dependant upon the clinical application and location of the stent. For instance, stents placed in vessels known to exhibit substantial pulsatility may require that the stent be designed to have end regions which are larger in diameter than the middle portion of the stent to better anchor the stent at the target location. Additionally, the width of the backbone may vary to provide regions of the stent which are less susceptible to elongation, thereby creating a stent that has localized stretch resistant properties which aids in reducing stent migration. Stents sufficient for treating an aneurysm without the aid of other embolization devices positioned within the aneurysm may require that the porosity of the deployed stent in the region of the aneurysm neck be less than about 30 percent. Additionally, stents for treating aneurysm in certain locations may require that the porosity across the neck be less than 30 percent however the porosity adjacent either side of the aneurysm neck be greater than 40 percent and have dimensions as not to occlude small perforator vessels adjacent the aneurysm neck. Stents used to treat fusiform aneurysms may be considerably longer than stents for berry aneurysms. Stents for use in treating a stenotic lesion may require more or less than 50 percent porosity however side member geometry should be designed to keep fragmented plaque trapped between the exterior wall of the stent and interior wall of the vessel.

As previously discussed, a specific stent device design is heavily dependant upon the clinical application for the device and may include materials or coatings to improve the biocompatibility of the device such as coatings that include ligands adapted to capture endothelial progenitor cells within the vasculature. Additionally, the stent device may include portions of the device such as side extension members which are formed of bio-erodible or bio-absorbable materials and or materials suitable for the delivery of pharmacological or therapeutic agents adapted to encourage healing during the treatment of aneurysms or reduction of plaque or restenosis during the treatment atherosclerotic lesions. Materials and coating process technology suitable for application to the present invention are described in U.S. Patent Application Publication No: 20070128723 A1 to Cottone et al., entitled, “Progenitor Endothelial Cell Capturing with a Drug Eluting Implantable Medical Device” herein incorporated by reference in it's entirety.

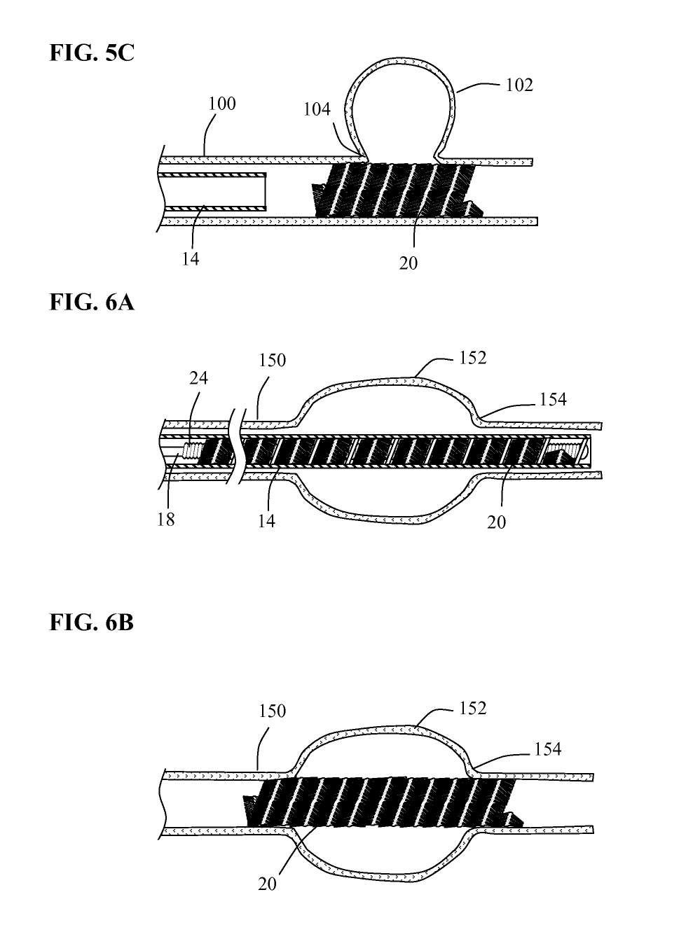

FIGS. 5A through 5C illustrate a method of deploying a stent device adjacent a vascular defect according to one embodiment of the present invention. The deployment system is positioned within a target vessel 100 having a bulging vascular defect known as an aneurysm 102. The interior of the aneurysm is coupled to the lumen of the vessel at aneurysm neck 104. The catheter distal end 14 including a stent device 20 is positioned adjacent aneurysm neck 104. Stent device 20, being in its first constrained configuration for delivery, is mounted on the distal end 24 of delivery member 18 and positioned within the gaps formed by spiral retainer member 22 and the inner wall of catheter distal end 14. As the catheter distal end 14 is retracted relative to delivery member 18, a portion of stent device 20 exits the distal end of the catheter and being formed from a resilient material moves from its first constrained configuration to its second expanded configuration where the stent device 20 contacts the inner wall of vessel 100. Further retraction of catheter distal end 14 relative to delivery member 18 allows more of stent device 20 to be deployed in a helical overlapping fashion. During the deployment process the amount of overlap between adjacent turns of stent device 20 may be modified by advancing or retracting delivery member 18 while retracting catheter 14. This process of modifying the overlap amount gives the physician the ability to increase or decrease the porosity of the stent device 20 as needed for a particular target deployment location. Deployment of stent device 20 is completed when the last portion of the stent device exits the lumen of catheter distal end 14 and all of stent device 20 is in its second expanded configuration.

Until the last portion of stent device 20 has been deployed, it may be possible to re-sheath stent device 20 by advancing catheter distal end 14 relative to delivery member 18. As catheter distal end 14 is advanced, previously deployed turns of stent device 20 will become re-constrained within the gap formed by spiral retainer member 22 around distal coil 24 and the inner wall of the catheter lumen. Once stent device 20 has been completely re-sheathed, the catheter distal end 14 may be repositioned within vessel 100 relative to aneurysm neck 104 for subsequent redeployment of stent device 20 according to aforementioned procedures.

FIGS. 6A and 6B illustrate a method of deploying a stent device adjacent a vascular defect according to another embodiment of the present invention. The deployment system is positioned within a target vessel 150 having a bulging vascular defect known as a fusiform aneurysm 152 which encompasses the circumference of the vessel. The interior of the aneurysm is coupled to the lumen of the vessel at aneurysm neck 154. The catheter distal end 14 having including stent device 20 is positioned adjacent aneurysm neck 104. Stent device 20, being in its first constrained configuration for delivery, is mounted on the distal end 24 of delivery member 18 and positioned within the gaps formed by spiral retainer member 22 and the inner wall of catheter distal end 14. As the catheter distal end 14 is retracted relative to delivery member 18, a portion of stent device 20 exits the distal end of the catheter and being formed from a resilient material moves from its first constrained configuration to its second expanded configuration where the stent device 20 contacts the inner wall of vessel 150. Further retraction of catheter distal end 14 relative to delivery member 18 allows more of stent device 20 to be deployed in a helical overlapping fashion until stent device 20 fully spans aneurysm neck 154.

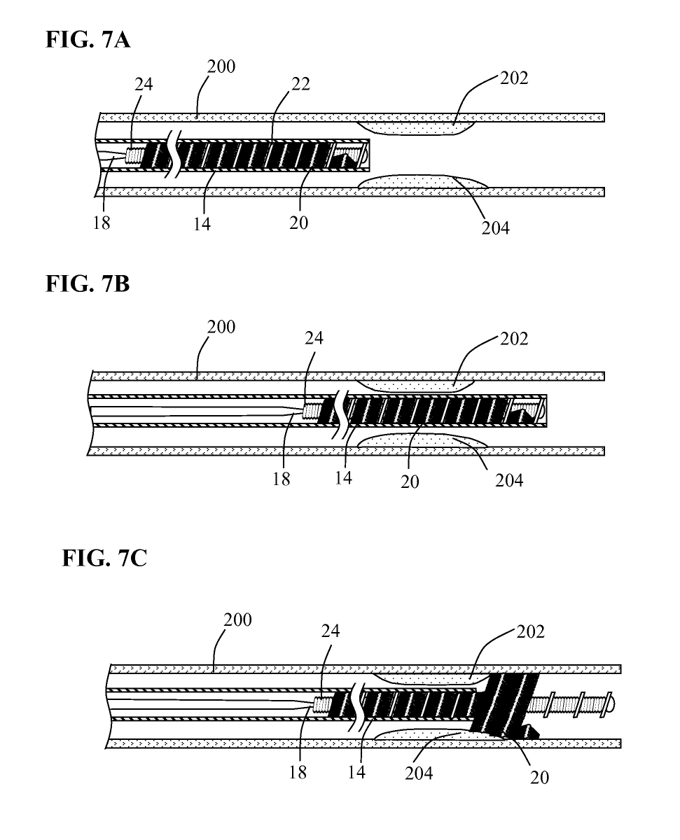

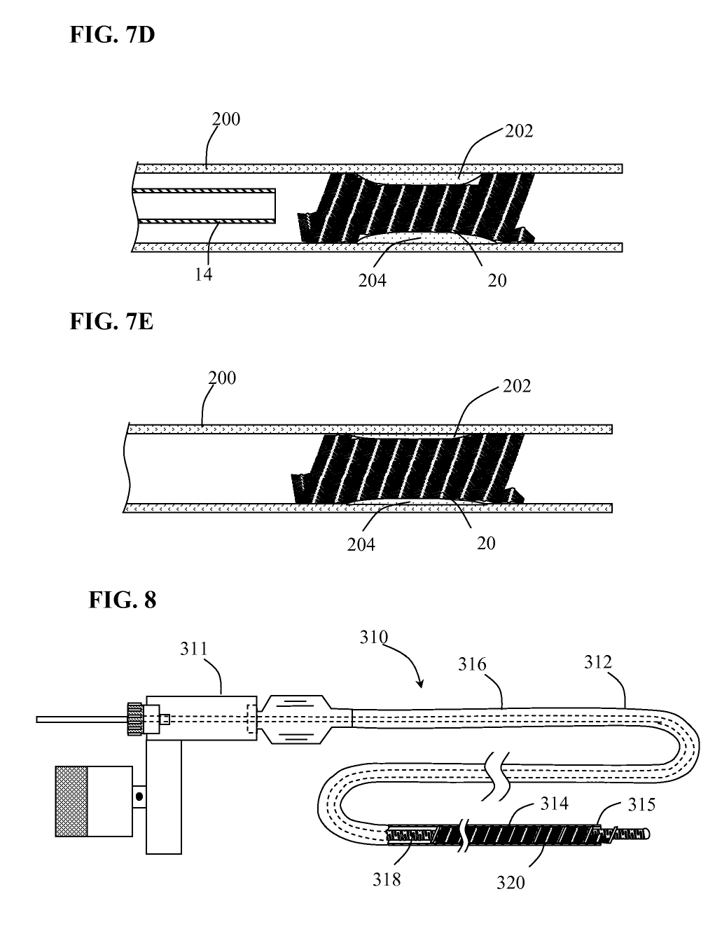

FIGS. 7A through 7E illustrate a method of deploying a stent device adjacent a vascular defect according to another embodiment of the present invention. The deployment system is positioned within a target vessel 200 having an atherosclerotic lesion comprising plaque deposits 202 and 204 creating a stenosis within the vessel restricting distal blood flow. The catheter distal end 14 including a stent device 20 is positioned adjacent aneurysm neck 104. Stent device 20, being in its first constrained configuration for delivery, is mounted on the distal end 24 of delivery member 18 and positioned within the gaps formed by spiral retainer member 22 and the inner wall of catheter distal end 14. As the catheter distal end 14 is retracted relative to delivery member 18, a portion of stent device 20 exits the distal end of the catheter and being formed from a resilient material such as nitinol moves from its first constrained configuration to its second expanded configuration where the stent device 20 contacts the inner wall of vessel 200 distal to the lesions. Further retraction of catheter distal end 14 relative to delivery member 18 allows more of stent device 20 to be deployed in a helical overlapping fashion contacting the plaque deposits 202 and 204. Deployment of stent device 20 is completed when the last portion of the stent device exits the lumen of catheter distal end 14 and all of stent device 20 is in its second expanded configuration spanning the lesions. Although stent device 20 is in the second expanded deployment configuration stent device 20 has a normal unconstrained diameter which is larger than the second expanded configuration and thusly the inner diameter of vessel 200. The resilient nature of stent device 20, being in an expanded configuration and slightly constrained by the lesion and vessel, creates chronic outward force which is applied to plaque deposits 202 and 204 as well as vessel 200. The chronic outward of force applied by the stent device 20 is a result of many different design attributes of the stent including the dimensions and geometry of the backbone, the phase transformation temperature, Af, of the nitinol used and the shape set normal unconstrained expanded diameter of the stent. When properly designed, the chronic outward force of stent device 20 allows the gradual expansion of the stent diameter in the vicinity of the plaque deposits 202 and 204 to thereby compress the plaque deposits thus reducing the restriction to blood flow in the region. Alternatively, a balloon device may be positioned within the lumen of the deployed stent device 20 and inflated to accelerate the compression of plaque deposit thereby permitting immediate revascularization.

FIG. 8 illustrates a medical device deployment system 310 suitable for use with embodiments of the present invention. System 310 includes a handle assembly 311, a catheter 312 having a distal end 314 with a tip marker 315 and a proximal end 316, an elongate delivery member 318, and a stent device 320 for use in vascular reconstruction or revascularization procedures.

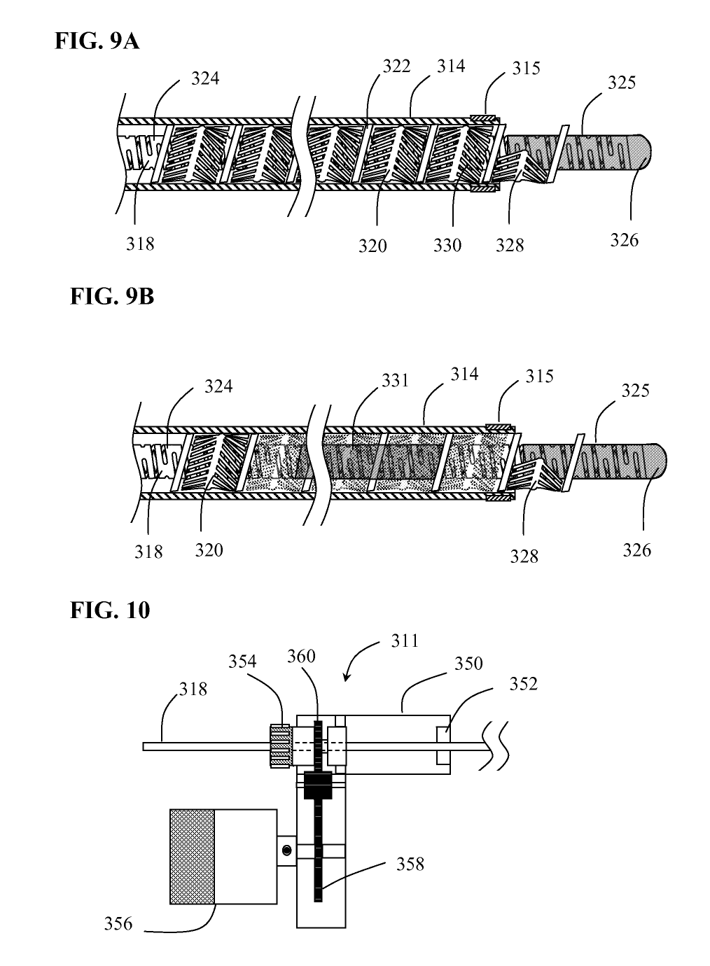

FIG. 9A depicts a magnified partial cross-sectional view of catheter distal end 314. Slidably positioned within the lumen of catheter 312 is delivery member 318. Delivery member 18 is preferably formed as an elongate assembly and includes at its distal end a raised spiral retainer member 322 secured to a flexible torque-able member 324. Flexible torque-able member 324 preferably takes the form of a laser cut hypo-tube and located at its distal end is delivery member tip marker 325 and an atraumatic tip 326. Tip marker 325 is preferably formed using shrink tubing but may be formed using standard marker banding techniques such as applying coils, rivets or crimped marker bands to provides visibility of the tip of the delivery member under fluoroscopy, magnetic resonance imaging and or direct visualization. As shown, atraumatic tip 326 may be formed as a rounded bead using beading processes known for guide wire manufacturing such as soldering, plasma or arc welding. Alternatively atraumatic tip 326 may be formed of a soft polymer extension to reduce the likelihood of vessel perforation. Spiral retainer member 322 preferably takes the form of a flexible wire which is fixedly secured to the outer surface of torque-able member 324 using known processes such as soldering welding or gluing. The pitch of spiral retainer member 322 creates a spiral gap relative to the outer surface of torque-able member 324 and the wall of catheter distal end 314. Also located at the distal end of delivery member 318 is stent device 320 which is generally formed as a helix includes a primary backbone 328 having a plurality of side extension members 330. Stent device 320 has a first constrained configuration for delivery to a target location and a second expanded configuration for deployment at a target site. In the first constrained configuration of stent device 320, primary backbone 328 and side extension members 330 are positioned within the spiral gap formed by spiral retainer member 322 and are constrained by the inner wall of catheter distal end 314.

FIG. 9B also depicts a magnified partial cross-sectional view of catheter distal end 314 with a portion of stent device 320 shown in dashed lines to illustrate positioning marker 331. Positioning marker 331 is preferably a radiopaque marker coupled to the distal end of the delivery member 318 proximal to tip marker 325, located beneath stent device 320 in a delivery configuration and correlates to a centered deployment location of the deployed stent device 320. Positioning marker 331 is preferably formed using shrink tubing but may be formed using standard marker banding techniques such as applying coils, rivets or crimped marker bands to provides visibility of the tip of the delivery member under fluoroscopy and or magnetic resonance imaging. The location and length of the positioning marker 331 depends upon the deployed length and diameter of stent device 320 and the inner diameter of catheter 312.

Also shown in FIG. 9B are catheter tip marker 315 and delivery member tip marker 325. Catheter tip marker 315 is preferably positioned at the distal tip of catheter 312 and is formed using known techniques for constructing and applying markers. Delivery member tip marker 325 extends to the distal spiral gap formed by retainer member 322. The relative positions of the catheter tip marker 315 and delivery member tip marker 325 illustrated in FIG. 9B indicate that as the delivery member tip marker is positioned distal to the catheter tip marker, the delivery member tip and the distal spiral gap formed by retainer member 322 exit the lumen of catheter 312 and that deployment of stent device 320 may commence.

FIG. 10 illustrates an attachable handle assembly 311 having a housing member 350, a catheter coupler 352 and a rotatable delivery member coupler 354 which couples to both the proximal end 316 of delivery catheter 312 and the proximal end of the delivery member 318. The handle assembly 311 further includes a rotatable deployment knob 356 connected to a gear assembly 358 which is coupled to the rotatable delivery member coupler 354 such that rotation of the deployment knob 356 causes the rotation of the rotatable delivery member coupler 354. The gear assembly 358 may be configured such that one rotation of the deployment knob 356 corresponds to multiple rotations of the rotatable delivery member coupler 354. The rotatable delivery member coupler 354 may be selectively secured to the proximal end of the delivery member such that rotation of the deployment knob 356 causes rotation of the delivery member 318. The rotatable delivery member coupler preferably takes the form of a rotating hemostasis valve (RHV) assembly, which can be securely coupled to the delivery member, having a gear member 360 that intermeshes with the gear assembly 358 coupled to the rotatable deployment knob.

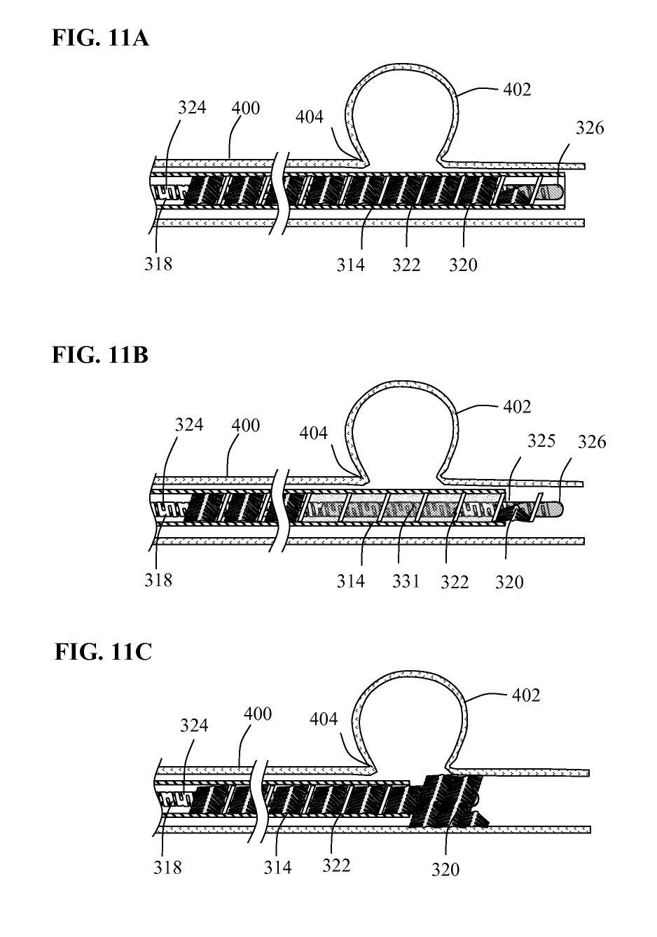

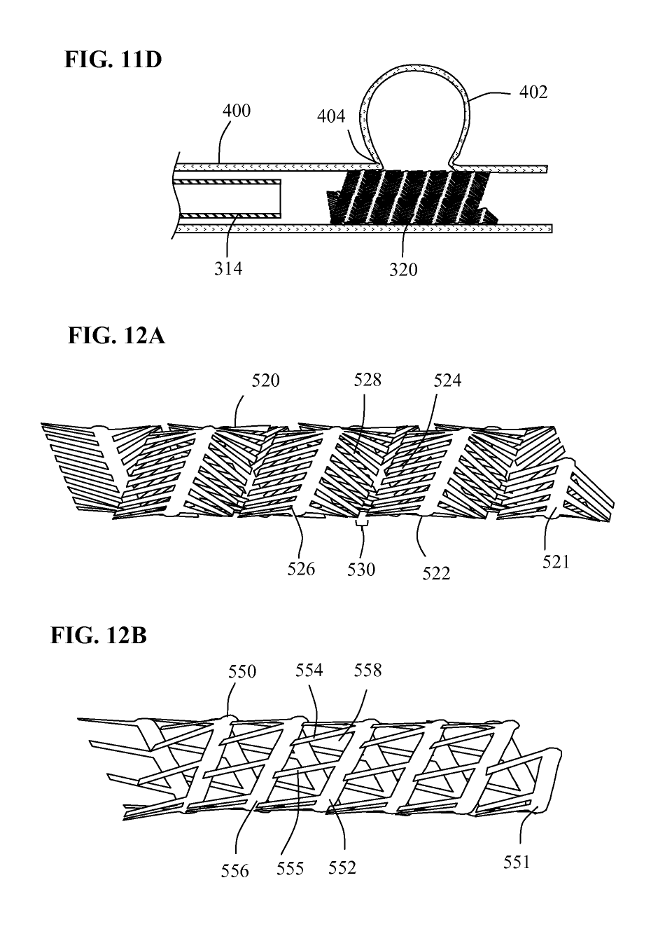

FIGS. 11A through 11D illustrate a method of deploying a stent device adjacent a vascular defect according to one embodiment of the present invention. The deployment system is positioned within a target vessel 400 having a bulging vascular defect known as an aneurysm 402. The interior of the aneurysm is coupled to the lumen of the vessel at aneurysm neck 404. The catheter distal end 314 including a stent device 320 is positioned adjacent aneurysm neck 404. Stent device 320, being in its first constrained configuration for delivery, is mounted on the distal end 324 of delivery member 318 and positioned within the gaps formed by spiral retainer member 322 and the inner wall of catheter distal end 314. Under fluoroscopic visualization the positioning marker 331 of delivery member 318 is centered beneath the aneurysm neck 404. The catheter proximal end 316 is then secured to the catheter coupler of the handle assembly. The proximal end of delivery member 318 is positioned through the rotatable delivery member coupler in an unsecured fashion. The catheter distal end 314 is retracted relative to delivery member 318 until delivery member tip marker 325 is positioned distal to catheter tip marker 315. A portion of stent device 320 exits the lumen at the distal end 314 of catheter 312 and being formed from a resilient material moves from its first constrained configuration to its second expanded configuration where the stent device 320 contacts the inner wall of vessel 400. The rotatable delivery member coupler is then secured to the proximal end of the delivery member 318. The deployment knob of the handle assembly is then rotated to cause the delivery member 318 and spiral retainer member 322 to rotate thus advancing stent device 320 thereby allowing more of stent device 320 to be deployed in a controlled helical overlapping fashion. During the deployment process the amount of overlap between adjacent turns of stent device 320 may be modified by advancing or retracting coupled catheter 312 and delivery member 318 while rotating deployment knob. This process of modifying the overlap amount gives the physician the ability to increase or decrease the porosity of the stent device 320 as needed for a particular target deployment location. Deployment of stent device 320 is completed when the last portion of the stent device exits the lumen of catheter distal end 314 and all of stent device 320 is in its second expanded configuration.

FIG. 12A illustrates detail of another embodiment of the present invention stent device 520 in an expanded configuration. The helical primary backbone 521 with a first wind 522 is shown along with a representative first wind side extension member 524 and adjacent wind 526 and representative adjacent wind side extension member 528. As depicted and previously described, the side extension members of the stent device generally have one end secured to the backbone and the other end uncoupled. Stent device 520 is shown with first wind side extension member 524 not overlapping adjacent wind side extension member 528. The non-overlap of these side extension members is represented by gap 530. The size, shape and distribution of the gap between the side extension members is dependant upon the size shape and distribution of the side extension members and adjacent wind side extension members and in part by the pitch of the primary backbone in addition to the method of stent deployment. FIG. 12B illustrates detail of stent device 550 according to another embodiment of the present invention where the primary backbone 551 has a generally uniform helical shape. A backbone first wind 552 is shown with representative first wind side extension members 554 and 555 overlapping a backbone adjacent wind 556. As shown the side extension members of this particular stent device 550 are one side of backbone 551. The overlapping nature of the side extension members and first and adjacent winds of the backbone define the boundary of aperture 558. With numerous side extension members, numerous apertures are created providing a pseudo closed cell appearance. Of course the size, shape and location of these apertures are dependant upon the size, shape and distribution of side extension members as well as the size, shape and configuration of the backbone.

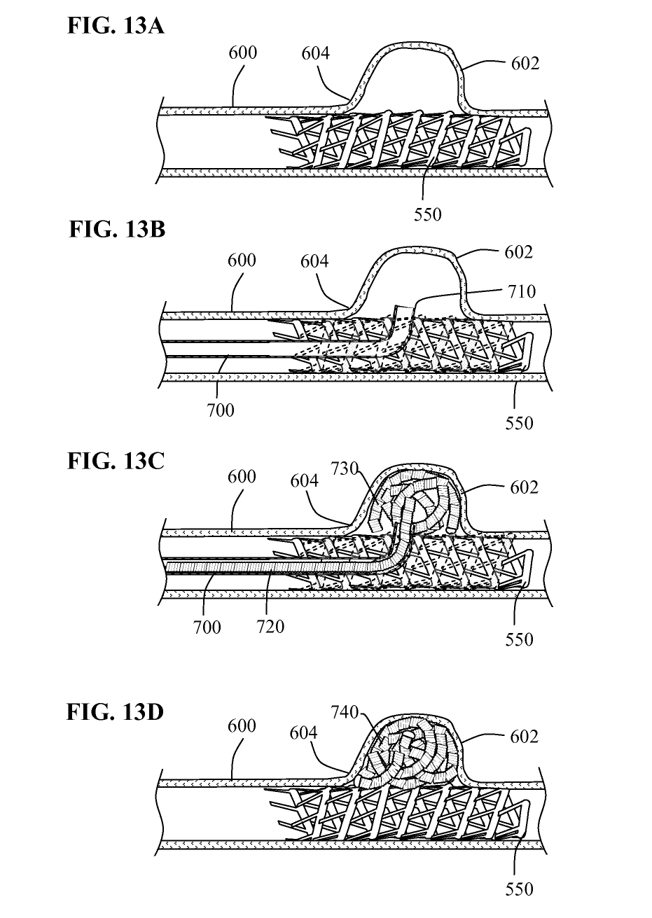

FIGS. 13A through 13D illustrate a method of using a stent device according to an embodiment of the present invention. FIG. 13A shows a vessel 600 having an aneurysm 602. Aneurysm 602 is shown having a wide aneurysm neck 604. Wide necked aneurysms typically have a geometry which is difficult to treat using embolic coils alone. Stent device 550, which has been advanced using a delivery system and any of the aforementioned delivery methods, is shown positioned within vessel 600 extending across aneurysm neck 604. FIG. 13B shows embolic delivery catheter 700 having being advanced adjacent the target site, positioned within vessel 600 with catheter tip 710 extending through an aperture of stent 550 and into aneurysm 602. FIG. 13C shows embolization coil 720 partially within the lumen of catheter 700 and partially within aneurysm 602. Previously detached embolic coils 730 are shown within aneurysm 602 being supported by the wall of stent device 550. FIG. 13D shows aneurysm 602 with a packed mass of embolization coils 740 being supported by stent 550 adjacent to aneurysm neck 604 and catheter 700 withdrawn from the vessel.

Novel devices, systems and methods have been disclosed to perform vascular reconstruction and revascularization procedures within a mammal. Although preferred embodiments of the invention have been described, it should be understood that various modifications including the substitution of elements or components which perform substantially the same function in the same way to achieve substantially the same result may be made by those skilled in the art without departing from the scope of the claims which follow.

What is claimed is:

1. An endolumenal reconstruction device for placement in a body lumen of a mammal comprising:
an elongate primary member formed of a resilient material having a generally coiled configuration with multiple adjacent turns defining a generally helical gap between said turns; and,
a plurality of side extension members having first and second ends wherein only one of said ends of each side extension member is coupled to said primary member, said side extension members extending outwardly from said primary member in a generally coplanar direction such that when said primary member is in said coiled configuration said side extension members at least partially span the helical gap of said turns.

2. A reconstruction device according to claim 1 wherein said side extension members are only fixedly coupled to each other through said primary member.

3. A reconstruction device according to claim 1 wherein said side extension members on a first turn of said primary member, overlap the side extension members on a second turn of said of said primary member.

4. A reconstruction device according to claim 1 wherein said side extension members on a first turn of said primary member, overlap a second turn of said primary member.

5. A reconstruction device according to claim 1 wherein said side extension members on a first turn of said primary member, are adjacent the side extension members on a second turn of said of said primary member.

6. A reconstruction device according to claim 1 wherein said side extension members on a first turn of said primary member, are adjacent a second turn of said primary member.

7. A reconstruction device according to claim 1 wherein at least one of said side extension members is arcuate.

8. A reconstruction device according to claim 1 wherein at least one of said side extension members comprises a marker.

9. A reconstruction device according to claim 1 wherein at least one of said side extension members has an end that is tabular.

10. A reconstruction device according to claim 1 wherein at least one of said side extension members includes an aperture.

11. A reconstruction device according to claim 1 wherein said primary member includes an aperture.

12. A reconstruction device according to claim 1 wherein said primary member includes a marker.

13. A reconstruction device according to claim 1 wherein said primary member has a first width at one portion of said primary member and a second width which is greater than said first width at another portion of said primary member.

14. A reconstruction device according to claim 1 wherein said side extension members along a portion of said primary member taper in length.

15. A reconstruction device according to claim 1 wherein the distribution of side extension members along a first portion of said primary member is greater than the distribution of said side extension members along a second portion of said primary member.

16. A reconstruction device according to claim 1 wherein a first coiled diameter of said primary member is greater than a second coiled diameter of said primary member.

17. A reconstruction device according to claim 1 wherein said side extension members are positioned on only one side of said primary member.

18. A reconstruction device according to claim 1 wherein said side extension members are positioned on opposite sides of said primary member.

19. A reconstruction device according to claim 1 wherein said primary member has a periodic arcuate shape in addition to said generally coiled configuration.

20. A stent device for placement in a body lumen of a mammal comprising:
an elongate primary member formed of a resilient material having a generally helical configuration with multiple adjacent turns said adjacent turns defining a generally helical gap between said turns; and,
a plurality of discrete side extension members having a first end region fixedly coupled to said primary member and a second end region extending outwardly from said primary member such that when said primary member is in said helical configuration said side extension members at least partially span a portion of said helical gap to thereby form a generally tubular framework.

21. A stent device according to claim 20 wherein said side extension members on a first turn of said primary member, overlap the side extension members on a second turn of said of said primary member.

22. A stent device according to claim 20 wherein said side extension members on a first turn of said primary member, overlap a second turn of said primary member.

23. A stent device according to claim 20 wherein said side extension members on a first turn of said primary member, are adjacent the side extension members on a second turn of said of said primary member.

24. A stent device according to claim 20 wherein said side extension members on a first turn of said primary member, are adjacent a second turn of said primary member.

25. A stent device according to claim 20 wherein at least one of said side extension members is arcuate.

26. A stent device according to claim 20 wherein at least one of said side extension members comprises a marker.

27. A stent device according to claim 20 wherein at least one of said side extension members has an end that is tabular.

28. A stent device according to claim 20 wherein at least one of said side extension members includes an aperture.

29. A stent device according to claim 20 wherein said primary member includes an aperture.

30. A stent device according to claim 20 wherein said primary member includes a marker.

31. A stent device according to claim 20 wherein said primary member has a first width at one portion of said primary member and a second width which is greater than said first width at another portion of said primary member.

32. A stent device according to claim 20 wherein said side extension members along a portion of said primary member taper in length.

33. A stent device according to claim 20 wherein the distribution of side extension members along a first portion of said primary member is greater than the distribution of said side extension members along a second portion of said primary member.

34. A stent device according to claim 20 wherein a first coiled diameter of said primary member is greater than a second coiled diameter of said primary member.

35. A stent device according to claim 20 wherein said side extension members are positioned on only one side of said primary member.

36. A stent device according to claim 20 wherein said side extension members are positioned on opposite sides of said primary member.

37. A stent device according to claim 20 wherein said primary member has a periodic arcuate shape in addition to said generally coiled configuration.

38. A stent implant system for repairing a body lumen comprising:
an elongate catheter having proximal and distal ends and a lumen extending therethrough;
an elongate torque-able delivery member having proximal and distal ends and being slidably positioned within the lumen of said catheter, said delivery member further including a spiral retainer member defining a spiral gap positioned about said delivery member distal end;
a stent device having a first constrained configuration for delivery to a target site within a body lumen and a second unconstrained configuration for deployment at a target site within the body lumen, said stent device being operable between said first and second configurations, said stent device comprising a primary member having a helical configuration with multiple turns and a plurality of extension members extending outwardly from said primary member, each extension member having first and second ends wherein one of said first and second ends is fixedly coupled to said primary member and the other end is uncoupled to the primary member of said stent device,
said stent device primary member being positioned in said spiral gap at the distal end of said delivery member disposed within the lumen of said catheter in a constrained said first configuration and extending from said spiral gap at the distal end of said delivery member upon exiting said catheter in an unconstrained said second configuration.

39. A medical device implant system for repairing a body lumen in a mammal comprising:
an elongate catheter member having proximal and distal ends and a lumen extending therethrough;
an elongate torque-able delivery member having proximal and distal ends and being slidably positioned within the lumen of said catheter, said delivery member further including a spiral retainer member defining a spiral gap positioned about said delivery member distal end, a radio-opaque tip marker located adjacent said delivery member distal end and a radio-opaque positioning marker located proximal to said tip marker;
a reconstruction device having a first constrained elongate tubular configuration for delivery to a target site within a body lumen and a second unconstrained expanded tubular configuration for deployment at a target site within the body lumen, said reconstruction device being operable between said first and second configurations, said reconstruction device also including a primary member having a helical shape and a plurality of extension members, each extension member having first and second ends wherein one of said first and second ends is fixedly coupled to said primary member and the other end is uncoupled to the primary member of said reconstruction device,
said reconstruction device primary member being positioned in said spiral gap at the distal end of said delivery member disposed within the lumen of said catheter in a constrained said first configuration and extending from said spiral gap at the distal end of said delivery member upon exiting said catheter in an unconstrained said second configuration; and,
a handle assembly having a housing, a catheter coupler, a selectively operable delivery member coupler and an actuator member, wherein said handle assembly is engaged with the proximal ends of said catheter and said delivery member and operation of said actuator member causes the rotation of said delivery member to thereby cause the reconstruction device to move from a catheter constrained first configuration to a catheter unconstrained second configuration.

40. A method of deploying an implant comprising the steps of:
positioning a stent deployment system including a catheter, a delivery member and a stent device having a coiled primary member and a plurality of discrete side extension members extending from said primary member within a vessel adjacent a target site;
retracting the catheter relative to a delivery member of said stent deployment system;
deploying a portion of said stent device adjacent the target site;
controlling the amount of overlap of the side extension members of the stent device during deployment of the stent adjacent the target site;
disengaging the stent device from the delivery member distal end and catheter lumen.
